(19) United States
(12) Patent Application Publication
(10) Pub. No.: US 2024/0423906 A1
(43) Pub. Date: Dec. 26, 2024

(54) SOLID IMPLANT FORMULATION FOR DRUG DELIVERY

(71) Applicant: Calista Therapeutics, Lincoln, RI (US)

(72) Inventor: Andrew P Mallon, Lincoln, RI (US)

(21) Appl. No.: 18/825,628

(22) Filed: Sep. 5, 2024

Related U.S. Application Data
(63) Continuation of application No. 17/465,748, filed on Sep. 2, 2021.
Application No. 17/465,748 is a continuation of application No. 16/818,918, filed on Mar. 13, 2020, now abandoned.
(60) Provisional application No. 62/818,559, filed on Mar. 14, 2019.

(51) Int. Cl. A61K 9/00; A61K 47/02

(52) U.S. Cl. CPC A61K 9/0024; A61K 47/02

(57) ABSTRACT

The present invention refers to a biodegradable delivery device that is implanted into patients to provide an extended release of one or more active pharmaceuticals for a therapeutic purpose. The delivery device comprises of: 1) an outer sheath that is composed of an inert biodegradable material that prevents the direct interactions of the core(s) with patient tissues and also facilitates the homogenous access of solvent to the surface area of the core to enable predictable core ingredient dissolution, depletion and safe and effective concentrations. 2) A Primary Core comprising one or more active ingredients plus excipients, including markers, that biodegrades and elutes all its ingredients before the sheath degrades. 3) A Secondary Core at the geometric center of the Primary core comprising a marker (including an active pharmacological or inactive ingredient that can be detected by bioanalysis, or a radio-opaque marker) that can signal when the secondary core is exposed, and excipients, which biodegrades and elutes its ingredients. 4) All elements have a marker excipient that renders the device observable remotely, including by x-ray detecting barium sulfate. The Secondary Core has a marker signal that can be distinguished from the Primary Core and Outer sheath.

### FIELD OF THE INVENTION

The present invention describes an implantable drug delivery device that will provide an extended release of one or more active ingredients and methods for its formulation and manufacture.

### BACKGROUND OF THE INVENTION

Many patients have difficulties maintaining compliance to critical medications over extended periods of time, especially patients that are highly susceptible to non-compliance.

### SUMMARY OF THE INVENTION

The present disclosure generally relates to a depot drug delivery device that is implanted into a patient to allow for sustained release of a drug or other pharmaceutically active material.

In addition, we describe methods of manufacture of this device that are unique and specialized to this formulation. Detailed illustration of this invention are found in FIG. 1.

In a preferred embodiment, the overall implant can vary in size, according to the active ingredient. The larger the surface area of the implant, the greater the resultant drug concentration. The surface area required will be a function of the therapeutic index of the ingredient, thereby achieving efficacy without safety. In addition, the dimensions of the implant, including but not limited to the diameter, will determine how long the depot will continue to dissolute until there is nothing left.

The invention also describes an outer sheath or coating that is made from any biodegradable or non-biodegradeable biostable and inert material that is obvious to those skilled in the art as having suitable properties.

In a particular embodiment, the outer sheath degrades more slowly than the core implant. Thereby, once the depot is depleted, the remaining sheath will also degrade. Importantly, the sheath material is chosen and delineated (thickness, concentration, density) in a manner that ensures it will outlast the depot and maintain its function until after the depot is depleted.

This attribute allows for one implanted depot to become depleted, then allow for a subsequent implant to be inserted, including at the same site.

This membrane has utility in separating the ingredients of the core from the tissues of the body into which the depot is implanted. This innovation prevents the development of localized toxicity reactions and provides additional structural integrity to the core.

The membrane has the additional utility in allowing for surface area to solvent interactions to occur homogenously across all or most of the implant. This solves the current problem that can occur whereby parts of the implant surface become inaccessible to solvent due to idiosyncratic positioning against tissues. The membrane is thick enough to allow solvent to move laterally through it and solvate the entire surface area of the implant.

The depot within the sheath can be susceptible to idiosyncratic breakdown that is a result of many expected and unexpected events that can lead to crumbling, fracturing, cracking and other events that alter the intended dissolution of the depot. This event can cause rapid loss of the active ingredient, causing dangerous spikes in concentration locally and systemically, endangering patients. In addition, the depot can become exhausted prematurely due to accelerated and/or sporadic dissolution, leading to failures in therapeutic efficacy and expose the patients to danger.

For example, these events can include: Implants being brittle, and cracking or fracturing; implants being poorly compressed, causing crumbling; palpations of the implant by patients or others; injury to the implant site; idiosyncratic placement of the implant in tissues; pathological events, like, but not limited to, inflammation, fibrosis and errors occurring during implantation.

In order to render the depot opaque, we describe the inclusion of a radio-opaque substance such as Barium Sulfate that can be added to the mixture prior to formulation of the implant. This can be incorporated into the sheath and the core. As the implant dissolves, the barium also dissolves. Observation of the implant using techniques such as x-ray, can both identify that a patient has an implant and provide information as to the size that is remaining.

A particular utility of this approach is that a subsequent administration of another implant ‘dose’ can be provided with knowledge about the prior implant site and the extent to which the prior depot or depots have degraded. This avoids the danger of overdose that occurs when a new implant is provided when the prior implant is not yet depleted. This also avoids the need to undertake blood test to elucidate this.

One particular drawback of using implants is a lack of knowledge about the state of the implant. We have propounded aspects of this invention that address this already. One skilled in the art may seek to determine whether there is a functional implant by testing patient plasma samples for presence of the suspected drug depot, However, this approach is unreliable because the amount of drug being released by a depot can be relatively consistent. Thereby, just by measuring drug concentrations, one would not know if an implant that was intended to last 6 months, for example, was at month 1,2,3,4 5 or 6 with any reliability.

The utility of opacity that we describe provides a reliable way to geometrically measure the mass of the implant and more accurately determine the time until depletion. Indeed, care-givers could repeatedly monitor size in order to track dissolution.

In certain embodiments, the active ingredient is a drug that in certain circumstances, become dangerous or interferes with the needs of the patients.

For example, an opioid use disorder patient treated with an implant containing Naltrexone®, the mu-opioid antagonist, would need, at some point, to have the implant removed. This could occur due to an adverse reaction, as it could for any ingredient; but it could also occur for a pharmacodynamic reason. For example, a patient needs opioid pain relief due to a condition that is painful. Opioids would have limited effectiveness and would be dangerous to intentionally overdose to try to out-compete the extant naltrexone antagonism.

Without the opacity of this invention, there are serious problems that cannot be easily overcome.

In one example, the patient may not be conscious, fully conscious or compus mentis, and thereby not in a position to inform caregivers about the implant. Caregivers could use a method such as x-ray to easily and obviously detect the presence and position and mass of an implant. This information would then be critical in informing them as to its safe extraction. In emergency situations, this would be of particular utility.

In another example, a patient may have an implant containing a psychiatric mediation for Schizophrenia, like Risperidone®. However, if adverse reactions arise, care givers would need to be able to remove the implant also. Or, the patient may inadvertently be treated by multiple care givers and unbeknownst, the correct doses may be administered doubled due to the implant, resulting in dangerous and distressing side effects.

In another embodiment, the implant core is a bi-core design. Within the geometric center of the implant, there is a small secondary core.

This secondary core is placed at the core and is of a size that provides a particular utility. As noted, the implant depot will last a predetermined duration. Under optimum conditions and implant dissolution behavior, the core will be progressively dissolved until the secondary core is exposed and also dissolves.

The secondary core is composed of a marker. This marker is a material that provides a signal that the core is exposed.

In a particular embodiment, a secondary core includes active ingredients that are pharmacologically commensurate with the primary core.

For example, a Naltrexone® primary core could have a Naloxone® secondary core, or a Risperidone® primary core could have an aripiprazole secondary core.

In a particular embodiment the core is comprised of a radio-opaque marker that is distinguishable from the primary core, or the primary core lacks the same marker.

In a particular embodiment, the core is comprised of a non-pharmacologically active marker that can be detected by bioanalysis or other methods.

The secondary core marker is a safe ingredient and may or may not have a pharmacological effect.

In a further embodiment, this secondary core marker aspect of the invention provides a particular utility. As described previously, the implant core can become prematurely exposed. In this situation, the depot can dissolve rapidly creating unintended concentrations higher than anticipated that are unsafe and potentially harmful to the patient, leading to adverse events, including serious adverse effects (AEs) in drugs that have a narrow therapeutic index between therapeutic efficacy and AEs.

The exposure of the secondary core will result in the dissolution of the core ingredients and their depletion.

In an embodiment where the primary and secondary core can be distinguished visually using observational methods, the observer will be able to see that the core is being depleted and that the structural integrity of the implant has prematurely failed.

In another embodiment, the blood can be sampled and tested for presence of the core ingredient using bioanalysis. The core ingredient providing a detectable signal.

This secondary core provides insight into the structural integrity of the implant and will inform caregivers as to decision for implant removal or replacement.

This aspect also provides safety monitoring utility to ensure safety, including but not limited to regulatory submissions for approval and post-approval monitoring.

This implant design can be used for any pharmaceutically active material or combinations of materials within one implant.

This invention further describes the method of formulation and administration of a therapeutic dose of the composition in a scheduled manner to elicit a therapeutic effect.

This invention also describes the manufacturing of this implant. This comprises multiple stages including the formulation of the secondary core, the primary core, and the outer sheath; each with its own ingredients.

In another aspect, this invention provides an administration kit for implantation of these devices in a manner that will maintain their integrity and ensure proper functionality.

### DETAILED DESCRIPTION OF THE INVENTION

Unless herein defined otherwise, all technical and scientific terms have the same meaning as is commonly understood by those of ordinary skill in the arts of this invention and as it is embodied and described. The following reference materials provide a general definition of many of the terms used in this invention: Singleton et al., Dictionary of Microbiology and Molecular Biology (2d ed. 1994); The Cambridge Dictionary of Science and Technology (Walker ed., 1988); and Hale & Marham, The Harper Collins Dictionary of Biology (1991). While any methods, known in the art, and materials comparable or equivalent to those described herein can be used in the practice or testing of the present invention, the preferred compositions, methods and materials are described explicitly.

Compounds of this invention include those described generally above, and are further described and drawn by the classes, subclasses, and species disclosed herein.

The dosage amount of a formulation of the present invention administered to an animal or patient can be determined by physical and physiological factors such as body weight, severity of condition, the type of disease being treated, previous or concurrent therapeutic interventions, idiopathy of the patient and on the manner and/or route of administration by a practitioner skilled in the art. The skilled practitioner(s) responsible for administration will, whatever the case may be, determine the concentration of active ingredient(s) in a composition and appropriate dose(s) for the individual subject.

In certain embodiments, pharmaceutical formulations of the inventive composition may comprise, for example, at least about 0.01% of an active compound(s). In other embodiments, the active inventive composition may comprise between about 2% to 100% of the weight of the unit, or between about 25% to about 60%, for example, and any range derivable therein.

Preferred dosage ranges include 0.001 to 20 μmol of composition per kg patient body weight. In other instances, the dosages range is from 0.005 to 5 μmol composition per kg patient body weight, optionally 0.01-5 μmol of composition per kg patient body weight. In some methods, 0.3 to 3 μmol composition per kg patient body weight are administered. In some methods, 0.1-1 μmol composition per kg patient body weight is administered, more preferably about 0.5 μmol composition per kg patient body weight. Dosage per kg body weight can be converted from rats to humans by dividing by 6.2 to compensate for different surface area to mass ratios. Dosages can be converted from units of moles to grams by multiplying by the molar weight of a composition. Suitable dosages of composition for use in humans can include 0.005 to 10 mg/kg patient body weight, or more preferably 0.02 to 5 mg/kg patient body weight or 0.1 to 1 mg/kg, or 0.2 to 0.9 mg/kg. In absolute weight for a 75 kg patient, these dosages translate to 0.075-375 mg, 0.375 to 75 mg or 7.5 mg to 75 mg or 12.5 to 67 mg. Rounded to encompass variations in e.g., patient weight, the dosage is usually within 0.5 to 500 mg, preferably 1 to 100 mg, 0.5 to 50 mg, or 1-20 mg.

In other non-limiting examples, a dose of the inventive composition and compositions may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body about weight, 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

The inventive composition may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

Objectives, features and advantages of the embodiments shall become apparent as the description thereof proceeds when considered in connection with the accompanying illustrative drawings.

### Indications for Use

Compositions of the present invention may have utility in therapeutic strategies intended to prevent, treat or cure one or more diseases, their symptoms and clinical sequelae thus arising.

In another preferred embodiments, the subject compounds may be administered to a patient suffering from pain and/or inflammation (for example, but not limited to, arthritis, retinopathy, SLE, psoriasis, Bullous pemphigoid, shingles or a similar condition), a subject at risk of, or having undergone, microvascular insufficiency, hypoxia, sub arachnoid hemorrhage, stroke, atherosclerosis or another acute or chronic neurological ischemic events patients with mild to severe traumatic brain injury, including diffuse axonal injury, hypoxic-ischemic encephalopathy and other forms of craniocerebral trauma, patients suffering from ischemic infarction, embolism and hemorrhage, e.g., hypotensive hemorrhage, subjects with neurodegenerative diseases including Alzheimer's disease, Lewy Body dementia, Parkinson's disease, Huntington's disease, multiple sclerosis, Nieman-Pick disease, diabetic neuropathy, glaucoma, macular degeneration (wet and dry AMD), retinitis pigmentosa, motor neuron disease, muscular dystrophy, peripheral neuropathies, metabolic disorders of the nervous system including glycogen storage diseases, and other conditions where neurons are damaged or destroyed, patients with abnormal immune activation, such as autoimmune Systemic Lupus Erythematosus, rheumatoid arthritis, Bullous pemphigoid, HIV-associated disorders, Type-I diabetes, and the like; while others may include those characterized by insufficient immune function. Other diseases that may be subject to treatment with compositions of the present invention include psychiatric disorders such as attention deficit hyperactive disorder, depression (in all forms), agoraphobia, bulimia, anorexia, bipolar disorder, anxiety disorder, autism, dementia, dissociative disorder, hypochondriasis, impulse control disorder, kleptomania, mood disorder, multiple personality disorder, chronic fatigue syndrome, insomnia, narcolepsy, schizophrenia, substance abuse, post-traumatic stress disorder, obsessive-compulsive disorder, and manic depression, radiation, chemical and biological agent damage, as well as complex disorders such as Gulf War Syndrome, or such-like syndromes. Compositions of the present invention can also be used to improve outcomes regarding addiction/addiction recovery. In certain embodiments, compounds of the present invention can also be used to decrease (e.g., inhibit) cell proliferation, including but not limited to cancer. Compositions may also be used to prevent, arrest, reverse and/or treat disorders of cognition, including but not limited to autism spectrum disorders, Down's Syndrome, Angelman Syndrome, Fragile X, Alzheimer's disease, schizophrenia and ADHD.

In another preferred embodiments, the subject compounds may be administered to a patient suffering from diseases of senescence, trauma or ischemic injuries to the brain, eye, ear, or spinal cord often produce permanent damage to neurons and cells. These injuries are serious medical problems with no effective pharmacological treatments. For example, ischemic cerebral stroke, sub-arachnoid hemorrhage or spinal cord injuries manifest themselves as acute loss of neurological capacity, encompassing small focal to global dysfunction, and sometimes leading to death. In vivo, a local decrease in CNS tissue vascular perfusion mediates neuronal death in both hypoxic and traumatic CNS injuries. Local ischemia is often caused by a disruption of the local vasculature, vessel thrombosis, vasospasm, or luminal occlusion by an embolic mass. This ischemia is widely understood to damage susceptible neurons disrupting a variety of cellular homeostatic mechanisms and triggering apoptopic and necrotic cell death signaling events.

In certain preferred embodiments, the present invention describes a method of treating or preventing a symptom associated with these diseases comprising administering an effective amount of a pharmaceutical composition to a subject in need thereof, wherein the pharmaceutical composition comprises any compound of the present invention.

### BRIEF DESCRIPTION OF THE DRAWING FIGURES

The drawing herein illustrate several exemplary embodiments of the present invention.

FIG. 1 shows an illustration of the invention, a bi-core, ensheathed drug delivery implant.

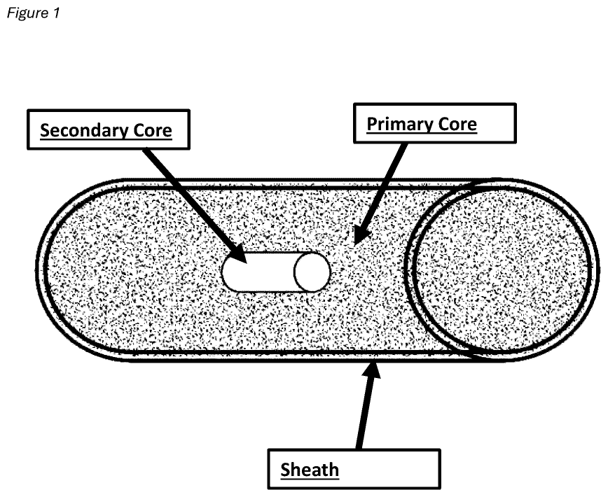

I claim:

1. An implanted biodegradable drug delivery device for the controlled release of one or more active pharmaceuticals over an extended period of time to produce local or systemic effects for a patient in need thereof, comprising:
a primary core comprising,
one or more pharmaceutical drugs; and
one or more excipients; and
a secondary core situated approximately at the center of the primary core comprising a marker.
an inert biodegradable outer sheath that separates the primary and secondary cores from a patient's tissues, wherein said sheath does not biodegrade or lose its functionality or structural integrity until after the core has been depleted.

2. The implanted biodegradable drug delivery device according to claim 1, wherein said one excipient causes the device to be opaque to x-ray observation procedures.

3. The implanted biodegradable drug delivery device according to claim 4, wherein said one excipient is barium sulfate.

4. The implanted biodegradable drug delivery device according to claim 1, wherein said marker indicates that the core is exposed.

5. The implanted biodegradable drug delivery device according to claim 1, wherein said marker comprises a pharmacodynamically equivalent, detectable ingredient.

6. The implanted biodegradable drug delivery device according to claim 7, wherein said pharmacodynamically equivalent, detectable ingredient is active.

7. The implanted biodegradable drug delivery device according to claim 7, wherein said pharmacodynamically equivalent, detectable ingredient is inactive.

8. The implanted biodegradable drug delivery device according to claim 7, wherein said marker can be detected using plasma bioanalysis.

9. The implanted biodegradable drug delivery device according to claim 7, wherein said pharmacodynamically equivalent, detectable ingredient is radio-opaque.

10. The implanted biodegradable drug delivery device according to claim 1, wherein said sheath further comprises a detectable marker.

11. The implanted biodegradable drug delivery device according to claim 12, wherein said sheath marker can be detected using plasma bioanalysis.

12. The implanted biodegradable drug delivery device according to claim 12, wherein said sheath marker is radio-opaque.

13. The implanted biodegradable drug delivery device according to claim 12, wherein the concentration of the markers in the secondary core and the sheath are different, further wherein said difference in concentrations can distinguish the secondary core marker from the sheath marker when the device is subjected to bioanalysis.
